(19) United States
(12) Patent Application Publication
(10) Pub. No.: US 2025/0256396 A1
(43) Pub. Date: Aug. 14, 2025

(54) System And Method For Performing Robot Registration Using Free-Form Path

(71) Applicant: MAKO Surgical Corp., Weston, FL (US)

(72) Inventors: Jevin Scrivens, Weston, FL (US); Huajin Qu, Fort Lauderdale, FL (US); Amar Bhatt, San Diego, CA (US)

(73) Assignee: MAKO Surgical Corp., Weston, FL (US)

(21) Appl. No.: 19/052,713

(22) Filed: Feb. 13, 2025

Related U.S. Application Data
(60) Provisional application No. 63/552,897, filed on Feb. 13, 2024.

(51) Int. Cl. B25J 9/16; A61B 34/00; A61B 34/20; A61B 34/30; B25J 5/00

(52) U.S. Cl. CPC B25J 9/1628; A61B 34/20; A61B 34/30; A61B 34/70; B25J 5/007; B25J 9/1679; A61B 2034/2055

(57) ABSTRACT

A surgical system includes a manipulator in a base coordinate system and a navigation system in a localizer coordinate system. The manipulator includes a tool and the navigation system includes a tool tracker. A control system controls movement of the tool and the tool tracker along a free-form path. The control system obtains a dataset related to movement of the tool tracker along the free-form path and obtains a dataset related to movement of the tool along the free-form path. The control system maps the datasets relative to one another to establish a relationship between the localizer coordinate system and the base coordinate system.

### CROSS-REFERENCE TO RELATED APPLICATIONS

The present application claims the benefit of and priority to U.S. Provisional Patent Application Ser. No. 63/552,897, filed Feb. 13, 2024, which is hereby incorporated by reference into the present application in its entirety.

### BACKGROUND

Robotic surgical systems, such as the MAKO® surgical robot, include a robotic arm that is supported by a base. An end effector is attached to the robotic arm. A base tracker is attached to the base of the robot and positionable via an adjustable support arm. The base tracker is detectable by a camera of a navigation system to track the position of the robot base.

“Robot registration” is a procedure for establishing a relationship between the base tracker and the base of the surgical robot. Robot registration is required, in part, due to the base tracker being adjustably set in any number of poses. Hence, the relationship between the base tracker and the base of the robot is unknown to the navigation system and must be determined using this procedure. In turn, this procedure confirms the accuracy of the robotic arm and the location of the cutting tool supported by the robotic arm, relative to the navigation system.

Conventionally, during robot registration, a user is prompted to move the robot arm and the tracker between vertices of a predefined cube to facilitate point collection from both the kinematics of the robot arm and tracker. These points are matched and compared throughout the robot registration process. However, during the registration process, the robotic system constrains movement of the robot arm to the predefined cube edges, thereby constraining the user's arm movements. There could also be obstructions to the robot arm or tracker along the cube edges. Additionally, movement along constrained cube edges can cause the camera to lose sight of the tracker. To avoid accuracy issues, the user must take the time to ensure the tracker is continuously visible throughout movement along the cube edges. Furthermore, the user must hold the robotic arm and the tracker static at each vertex of the cube for a threshold amount of time to ensure enough data is collected. This can add additional time to the registration process. Although the MAKO® registration process is robust, there is remains room for improvement in view of at least some of the challenges described above.

### SUMMARY

This Summary introduces a selection of concepts in a simplified form that are further described below in the Detailed Description below. This Summary is not intended to limit the scope of the claimed subject matter nor identify key features or essential features of the claimed subject matter.

In a first aspect, a surgical system is provided. The surgical system comprises a manipulator including: a base establishing a base coordinate system; a robotic arm coupled to the base and being formed of links and joints; and a tool coupled to the robotic arm; a navigation system including: a base tracker coupled to the manipulator; a tool tracker configured to be coupled to the tool; and a localizer configured to track positions of the base tracker and the tool tracker in a localizer coordinate system; and a control system coupled to the manipulator and the navigation system and being configured to: control, with the robotic arm, movement of the tool and the tool tracker along a free-form path; obtain, from the localizer, a localizer dataset related to the tool tracker and being derived from movement of the tool tracker along the free-form path; obtain, from the manipulator, a kinematic dataset related to the tool and being derived from movement of the tool along the free-form path; and map the localizer dataset and the kinematic dataset relative to one another to establish a relationship between the localizer coordinate system and the base coordinate system.

In a second aspect, a surgical system is provided. The surgical system comprises a manipulator including: a base establishing a base coordinate system; an arm coupled to the base and being formed of links and joints; and a tool coupled to the arm; a navigation system including: a base tracker coupled to the manipulator; a tool tracker configured to be coupled to the tool; and a localizer configured to track positions of the base tracker and the tool tracker in a localizer coordinate system; and a control system coupled to the manipulator and the navigation system and being configured to: obtain, from the localizer, a localizer dataset related to the tool tracker and being derived from movement of the tool tracker along the free-form path; obtain, from the manipulator, a kinematic dataset related to the tool and being derived from movement of the tool along the free-form path; and map the localizer dataset and the kinematic dataset relative to one another to establish a relationship between the localizer coordinate system and the base coordinate system.

In a third aspect, a surgical system is provided. The surgical system comprises a manipulator including: a base establishing a base coordinate system; a robotic arm coupled to the base and being formed of links and joints; and a tool coupled to the robotic arm; a navigation system including: a base tracker coupled to the manipulator; a tool tracker coupled to the tool; and a localizer configured to track positions of the base tracker and the tool tracker in a localizer coordinate system; and a control system coupled to the manipulator and the navigation system and being configured to: control, with the robotic arm, automatic movement of the tool and the tool tracker along a predetermined path; obtain, from the localizer, a localizer dataset related to the tool tracker and being derived from automatic movement of the tool tracker along the predetermined path; obtain, from the manipulator, a kinematic dataset related to the tool and being derived from automatic movement of the tool along the predetermined path; and map the localizer dataset and the kinematic dataset relative to one another to establish a relationship between the localizer coordinate system and the base coordinate system.

In a fourth aspect, a method of operating the surgical system of the first aspect is provided.

In a fifth aspect, a method of operating the surgical system of the second aspect is provided.

In a sixth aspect, a method of operating the surgical system of the third aspect is provided.

Any of the aspects above can be combined in part or in whole. Any of the aspects above can be combined in part or in whole with any of the following implementations. In some implementations, the manipulator comprises a cart that supports the robotic arm; the base coordinate system is established relative to the cart; and the base tracker is coupled to the cart by an adjustable support arm that extends from the cart.

In some implementations, the control system is configured to control, with the robotic arm, movement of the tool and the tool tracker in response external force applied to the tool by a user. In some implementations, the control system is further configured to control, with the robotic arm, movement of the tool and the tool tracker in a free mode whereby movement of the manipulator is unconstrained by a virtual boundary.

In some implementations, the free-form path is defined during movement of the tool and the tool tracker by the user, and the free-form path is unknown prior to movement of the tool and the tool tracker. In some implementations, the free-form path is defined by continuous movement of the tool and the tool tracker by the user and without requiring the tool and the tool tracker to be held static by the user at predetermined positions defined by the control system.

In some implementations, the surgical system further includes a display device and the control system is configured to provide, on the display device, an instruction or suggestion for the user for moving the tool.

In some implementations, the control system maps the localizer dataset and the kinematic dataset relative to one another by further being configured to: obtain, from the localizer dataset, a plurality of localized positions of the tool tracker being derived from movement of the tool tracker along the free-form path; obtain, from the kinematic dataset, a plurality of kinematic positions of the tool being derived from movement of the tool along the free-form path; and map the localized positions and kinematic positions relative to one another to establish the relationship between the localizer coordinate system and the base coordinate system. In some implementations, the control system maps the localizer dataset and the kinematic dataset relative to one another by further being configured to: obtain, from the localizer dataset, a localized path related to the tool tracker and being derived from movement of the tool tracker along the free-form path; obtain, from the kinematic dataset, a kinematic path related to the tool and being derived from movement of the tool along the free-form path; and map the localized path and the kinematic path relative to one another to establish the relationship between the localizer coordinate system and the base coordinate system. In some implementations, the control system is further configured to: obtain the localizer dataset according to a first sampling rate; and obtain the kinematic dataset according to a second sampling rate being different than the first sampling rate; and map the localizer dataset and the kinematic dataset relative to one another to offset a latency between the first sampling rate and the second sampling rate. In some implementations, the first sampling rate exhibits the latency relative to the second sampling rate.

In some implementations, the control system establishes the relationship between the localizer coordinate system and the base coordinate system by being configured to: obtain a first relationship between the localizer coordinate system and the tool tracker from the localizer; obtain a second relationship between the tool tracker and the tool from predetermined data; obtain a third relationship between the tool and the base coordinate system from kinematic data of the manipulator; and combine the first, second, and third relationships. In some implementations, the control system is further configured to: obtain a fourth relationship between the base tracker and the localizer coordinate system from the localizer; and combine the first, second, third, and fourth relationships to establish a fifth relationship between the base tracker and the base coordinate system.

In some implementations, the base coordinate system is determinable relative to the robotic arm; and the base tracker is coupled to the robotic arm and is moveable with the robotic arm. In some implementations, the control system is configured to: obtain a predetermined relationship between the base tracker and the base coordinate system from kinematic data related to the manipulator; and compare the established fifth relationship between the base tracker and the base coordinate system with the predetermined relationship between the base tracker and the base coordinate system to verify accuracy of the established fifth relationship.

### BRIEF DESCRIPTION OF THE DRAWINGS

Other advantages of the present disclosure will be readily appreciated, as the same becomes better understood by reference to the following detailed description when considered in connection with the accompanying drawings.

FIG. 1 is a perspective view of a robotic surgical system including a localizer, a manipulator, a surgical tool, and a tool tracker.

FIG. 2 is a block diagram of controllers of the robotic surgical system of FIG. 1.

FIG. 3 is a schematic view illustrating registration of the robotic surgical system.

FIG. 4 is a flowchart illustrating a method of path registration.

FIG. 5A is a diagram illustrating the surgical tool and the tool tracker of FIG. 1 moving along a free-form path during the method of path registration of FIG. 4.

FIG. 5B is a diagram illustrating a localized dataset generated by the localizer of FIG. 1 and a kinematic dataset generated by the manipulator of FIG. 1 based on movement of the surgical tool and the tool tracker of FIG. 1 moving along the free-form path.

FIG. 5C is a diagram illustrating a portion of a localized dataset generated by the localizer of FIG. 1 and a portion of a kinematic dataset generated by the manipulator of FIG. 1 in an instance where the localized dataset is generated according to a first sampling rate and the kinematic dataset is generated according to a second sampling rate.

FIG. 5D is a diagram illustrating mapping of the portion of the localized dataset of FIG. 5C and projected positions of the kinematic dataset of FIG. 5C.

### DETAILED DESCRIPTION

### I. System Overview

With reference to the Figures, wherein like numerals indicate like or corresponding parts throughout the several views, a surgical system 10 (hereinafter “system”) and method for operating the system 10 are described herein and shown throughout the accompanying Figures.

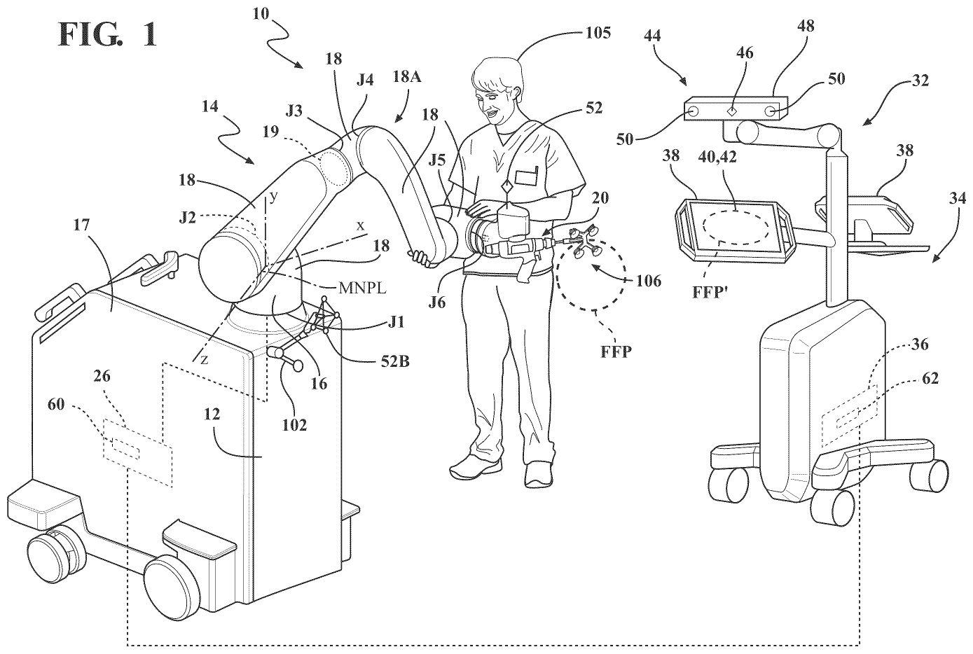

As shown in FIG. 1, the system 10 is a robotic surgical system for treating an anatomy (surgical site) of a patient 12, such as bone or soft tissue. In FIG. 1, the patient 12 is undergoing a surgical procedure. The anatomy in FIG. 1 includes a spine of the patient 12. The surgical procedure may involve tissue removal or treatment. The robotic surgical system 10 described herein may be utilized for treating any anatomical structure(s), for example, such as joints, including knee joints, hip joints, shoulder joints, ankles joints, or any other bone structure(s) not described herein. The robotic surgical system 10 can be used to perform any type of procedure, including any spinal procedure, partial knee arthroplasty, total knee arthroplasty, total hip arthroplasty, anatomical shoulder arthroplasty, reverse shoulder arthroplasty, fracture repair surgery, osteotomies, and the like. The techniques described herein can be used with any type of robotic system and for any procedure.

The system 10 includes a manipulator 14, which may also be referred to as a robotic manipulator. In one example, the manipulator 14 has a base 16 and plurality of links 18. The plurality of links 18 may be commonly referred to as a robotic arm 18A. In some instances, the manipulator 14 may include more than one robotic arm 18A. A manipulator cart 17 supports the manipulator 14 such that the manipulator 14 is fixed to the manipulator cart 17. The links 18 collectively form one or more arms of the manipulator 14. The manipulator 14 may have a serial arm configuration (as shown in FIG. 1) or a parallel arm configuration. In other examples, more than one manipulator 14 may be utilized in a multiple arm configuration. The manipulator 14 comprises a plurality of joints (J) and a plurality of joint encoders 19 located at the joints (J) for determining position data of the joints (J). For simplicity, one joint encoder 19 is illustrated in FIG. 1, although it is to be appreciated that the other joint encoders 19 may be similarly illustrated. The manipulator 14 according to one example has six joints (J1-J6) implementing at least six-degrees of freedom (DOF) for the manipulator 14. However, the manipulator 14 may have any number of degrees of freedom and may have any suitable number of joints (J) and redundant joints (J). In one example, each of the joints (J) of the manipulator 14 are actively driven and may be motorized joints (J). In other examples, each of the joints (J) may be passively driven. In still other examples, the joints (J) may include a combination of actively driven joints (J) and passively driven joints (J).

The base 16 of the manipulator 14 is generally a portion of the manipulator 14 that is stationary during usage thereby providing a fixed reference coordinate system (i.e., a virtual zero pose) for other components of the manipulator 14 or the system 10 in general. Generally, the origin of a base coordinate system is defined at the fixed reference of the base 16. The base coordinate system may be referred to herein as a manipulator coordinate system MNPL. The fixed reference point of the base 16 may be defined with respect to any suitable portion of the manipulator 14, such as one or more of the links 18. Alternatively, or additionally, the fixed reference point of the base 16 may be defined with respect to the manipulator cart 17, such as where the manipulator 14 is physically attached to the cart 17. In one example, the fixed reference point of the base 16 is defined at an intersection of the axes of joints J1 and J2. Thus, although joints J1 and J2 are moving components in reality, the intersection of the axes of joints J1 and J2 is nevertheless a virtual fixed reference point, which does not move in the manipulator coordinate system MNPL. The manipulator 14 and/or manipulator cart 17 house a manipulator computer 26, or other type of control unit.

The system 10 may include a surgical tool 20 coupled to the robotic arm 18A. The surgical tool 20 may include any end effector suitable for a surgical procedure. In some instances, the surgical tool 20 may be a tool for manipulating the anatomy of a patient, such as a saw, a router, a reamer, an impactor, an ultrasonic aspirator, a probe, a cutting tool, a drill, a dilator, a screwdriver, or the like. Additionally, or alternatively, the surgical tool 20 may include an accessory and/or energy applicator, such as a saw blade, a cutting burr, a router, a reamer, an impactor, an ultrasonic aspirator, a probe, a cutting tool, a drill, a dilator, a screwdriver, or the like. The accessory and energy applicator may be integrated or separately attached to surgical tool 20. The surgical tool 20 may also include a cutting guide. In some instances, the surgical tool 20 may include a tool holder or a guide tube for holding a tool, such as the tool holder/guide tube described in U.S. Provisional Patent Application No. 63/612,011, entitled, “Magnetic Spine Registration Tool”, which is incorporated herein by reference. The tool holder/guide tube and the robotic arm 18A may be separate components (i.e., two pieces) or the tool holder/guide tube and the robotic arm 18A may be integral with one another (i.e., one piece).

The system 10 may include a tool tracker 106. In one example, the tool tracker 106 may be temporarily coupled to the surgical tool 20, such as the trackable array described in U.S. Pat. App. Pub. No. 2022/0134569, entitled, “Robotic Surgical System With Motorized Movement To A Starting Pose For A Registration Or Calibration Routine,” the disclosure of which is hereby incorporated by reference, or such as the end effector tracker described in U.S. Pat. No. 10,350,012, entitled, “Method And Apparatus For Controlling A Haptic Device,” the disclosure of which is hereby incorporated by reference, or such as the tool tracker in U.S. Provisional Patent Application No. 63/612,011, entitled, “Magnetic Spine Registration Tool”, which is incorporated herein by reference. In other examples, the tool tracker 106 may be attachable to or detachable from the surgical tool 20 and/or attachable to or detachable from any other component of the manipulator 14, such as one or more links of the robotic arm 18A, e.g. a distal-most link of the manipulator (J6). For instance, the tool tracker 106 may include similar components as the tracker assembly described in U.S. Pat. App. Pub. No. 2023/0277256, entitled, “Robotic System Including A Link Tracker,” the disclosure of which is hereby incorporated by reference, for attaching the tool tracker 106 to the surgical tool 20 or any other component of the manipulator 14. For instance, the tool tracker 106 may be attached/detached to the surgical tool 20 or any other component of the manipulator 14 using a spring-biased latch, a magnetic connection, a snap-fit connection using flexible elements, or the like.

The tool tracker 106 may be coupled to the surgical tool 20 such that a relationship between the tool tracker 106 and the surgical tool 20 may be determinable. For example, the tool tracker 106 may include a reference surface configured to abut the surgical tool 20, such as the reference surface described in U.S. Provisional Patent Application No. 63/612,011, entitled, “Magnetic Spine Registration Tool”, which is incorporated herein by reference. Contact between the reference surface and the surgical tool 20 may indicate that the tool tracker 106 is properly coupled to the surgical tool 20 such that a location of the tool tracker 106 relative to the surgical tool 20 is fixed and that a relationship between the tool tracker 106 and the surgical tool 20 is determinable.

The tool tracker 106 may include one or more fiducial markers FM. In some instances, the fiducial markers FM may be coupled to or integrally formed with or manually coupled to the surgical tool 20 and/or a component of the manipulator 14. The fiducial markers FM may include any suitable shape. For example, the fiducial markers FM may include a cuboidal or spherical shape. The fiducial markers FM may be active or passive tracking elements.

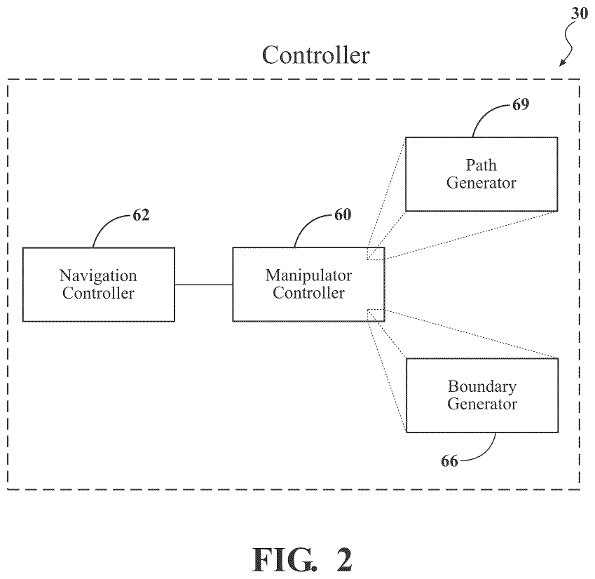

Referring to FIG. 2, the system 10 includes one or more controllers 30 (hereinafter referred to as “controller”). The controller 30 includes software and/or hardware for controlling the manipulator 14. The controller 30 directs the motion of components of the manipulator 14, such as the robotic arm 18A, and controls a state (position and/or orientation) of the surgical tool 20 with respect to a coordinate system. In one example, the coordinate system is the manipulator coordinate system MNPL, as shown in FIG. 1. The manipulator coordinate system MNPL has an origin located at any suitable pose with respect to the manipulator 14. Axes of the manipulator coordinate system MNPL may be arbitrarily chosen as well. Generally, the origin of the manipulator coordinate system MNPL is defined at the fixed reference point of the base 16. One example of the manipulator coordinate system MNPL is described in U.S. Pat. No. 9,119,655, entitled, “Surgical Manipulator Capable of Controlling a Surgical Instrument in Multiple Modes,” the disclosure of which is hereby incorporated by reference.

As shown in FIG. 1, the system 10 further includes a navigation system 32. One example of the navigation system 32 is described in U.S. Pat. No. 9,008,757, filed on Sep. 24, 2013, entitled, “Navigation System Including Optical and Non-Optical Sensors,” hereby incorporated by reference. The navigation system 32 is configured to track movement of various objects. Such objects include, for example, the manipulator 14, the surgical tool 20, and/or the anatomy. The navigation system 32 tracks these objects to gather state information of one or more of the objects with respect to a (navigation) localizer coordinate system LCLZ. Coordinates in the localizer coordinate system LCLZ may be transformed to the manipulator coordinate system MNPL, and/or vice-versa, using transformation and registration techniques described herein.

The navigation system 32 can include a cart assembly 34 that houses a navigation computer 36, and/or other types of control units. A navigation interface is in operative communication with the navigation computer 36. The navigation interface includes one or more displays 38. The navigation system 32 is capable of displaying a graphical representation of the relative states of the tracked objects to the operator using the one or more displays 38. First and second input devices 40, 42 may be used to input information into the navigation computer 36 or otherwise to select/control certain aspects of the navigation computer 36. As shown in FIG. 1, such input devices 40, 42 include interactive touchscreen displays. However, the input devices 40, 42 may include any one or more of a keyboard, a mouse, a microphone (voice-activation), gesture control devices, head-mounted devices, and the like.

The navigation system 32 is configured to depict a visual representation of the anatomy and the manipulator 14, and/or surgical tool 20 for visual guidance of any of the techniques described. The visual representation may be real (camera) images, virtual representations (e.g., computer models), or any combination thereof. The visual representation can be presented on any display viewable to the surgeon, such as the displays 38 of the navigation system 32, head mounted devices, or the like. The representations may be augmented reality, mixed reality, or virtual reality.

The navigation system 32 also includes a navigation localizer 44 (hereinafter “localizer”) coupled to the navigation computer 36. In one example, the localizer 44 is an optical localizer and includes a camera unit 46. The camera unit 46 has an outer casing 48 that houses one or more optical sensors 50.

The navigation system 32 may include one or more trackers. In one example, the trackers include the tool tracker 106, a pointer tracker, one or more manipulator trackers 52, and/or one or more patient trackers. In the illustrated example of FIG. 1, the manipulator tracker 52 is attached to a distal flange of the robotic arm 18A. The manipulator tracker 52 may be affixed to any suitable component of the manipulator 14, in addition to, or other than the surgical tool, such as the base 16 (i.e., tracker 52B), or any one or more links 18 or joints J of the manipulator 14. Additionally, or alternatively, the manipulator tracker 52 may be secured to a surgical drape or drape assembly, as described in U.S. Pat. App. Pub. No. 2023/0277256, entitled, “Robotic System Including A Link Tracker,” the disclosure of which is hereby incorporated by reference. For instance, the manipulator tracker 52 may be secured to a surgical drape or drape assembly via an elastic band or snap ring. The patient trackers may be affixed to a vertebra of the patient 12 and/or the pelvis of the patient 12. The pointer tracker may be affixed to a pointer used for registering the anatomy to the localizer coordinate system LCLZ. The trackers may be fixed to their respective components in any suitable manner.

As shown in FIG. 1, the base tracker 52B may be coupled to the cart 17 by an adjustable support arm 102. As shown, the base tracker 52B may be attached to one end of an adjustable support arm 102 and the adjustable support arm 102 may be attached at the other end to the cart 17. The adjustable support arm 102 can be positioned and locked to place the base tracker 52B in a fixed position relative to the cart 17. An example of a base tracker 52B coupled to an adjustable support arm can be like that described in U.S. patent application Ser. No. 17/513,324, entitled, “Robotic Surgical System With Motorized Movement To A Starting Pose For A Registration Or Calibration Routine”, or U.S. patent application Ser. No. 18/198,938, entitled, “Robotic System With Improved Configurations For Base Tracker”, the entire contents of which are hereby incorporated by reference in their entirety. Alternatively, or additionally, a base tracker 52B may be coupled to the robotic arm 18A and may be moveable with the robotic arm 18A. For instance, the base tracker 52B may include a plurality of (active or passive) tracking elements located on any number of links 18 of the manipulator 14. In this case, the base tracker 52B is formed of a tracking geometry from the various tracking elements, which move with movement of the robotic arm 18A. An example of a base tracker 52B formed by optical markers located on the links 18 may be like that described in U.S. patent application Ser. No. 18/115,964, entitled, “Robotic System with Link Tracker”, the entire contents of which is hereby incorporated by reference in its entirety. Alternatively, or additionally, the base tracker 52B may be secured to a surgical drape or drape assembly, as described in U.S. Pat. App. Pub. No. 2023/0277256, entitled, “Robotic System Including A Link Tracker,” the disclosure of which is hereby incorporated by reference. For instance, the base tracker 52B may be secured to a surgical drape or drape assembly via an elastic band or snap ring.

When optical localization is utilized, however, one or more of the trackers may include active markers 58. The active markers 58 may include light emitting diodes (LEDs). Alternatively, the trackers described herein may have passive markers, such as reflectors, which reflect light emitted from the camera unit 46. Other suitable markers not specifically described herein may be utilized.

The localizer 44 tracks the trackers to determine a state of one or more of the trackers which correspond respectively to the state of the object respectively attached thereto. The localizer 44 provides the state of the trackers to the navigation computer 36. In one example, the navigation computer 36 determines and communicates the state the trackers to the manipulator computer 26. As used herein, the state of an object includes, but is not limited to, data that defines the position and/or orientation of the tracked object or equivalents/derivatives of the position and/or orientation. For example, the state may be a pose of the object, and may include linear data, and/or angular velocity data, and the like.

Although one example of the navigation system 32 is shown in the Figures, the navigation system 32 may have any other suitable configuration for tracking the manipulator 14 and the patient 12. The illustrated tracker configuration is provided merely as one example for tracking objects within the operating space. Any number of trackers may be utilized and may be located in positions or on objects other than shown. In other examples, such as described below, the localizer 44 may detect objects absent any trackers affixed to objects.

In one example, the navigation system 32 and/or localizer 44 are ultrasound-based. For example, the navigation system 32 may comprise an ultrasound imaging device coupled to the navigation computer 36. The ultrasound imaging device may be robotically controlled or may be hand-held. The ultrasound imaging device images any of the aforementioned objects, e.g., the manipulator 14 and the patient 12, and generates state signals to the controller 30 based on the ultrasound images. The ultrasound images may be of any ultrasound imaging modality. The navigation computer 36 may process the images in near real-time to determine states of the objects. Ultrasound tracking can be performed absent the use of trackers affixed to the objects being tracked. The ultrasound imaging device may have any suitable configuration and may be different than the camera unit 46 as shown in FIG. 1. One example of an ultrasound tracking system can be like that described in U.S. patent application Ser. No. 15/999,152, filed Aug. 16, 2018, entitled “Ultrasound Bone Registration With Learning-Based Segmentation And Sound Speed Calibration,” the entire contents of which are incorporated by reference herein.

In another example, the navigation system 32 and/or localizer 44 are radio frequency (RF)-based. For example, the navigation system 32 may comprise an RF transceiver coupled to the navigation computer 36. The manipulator 14 and the patient 12 may comprise RF emitters or transponders attached thereto. The RF emitters or transponders may be passive or actively energized. The RF transceiver transmits an RF tracking signal and generates state signals to the controller 30 based on RF signals received from the RF emitters. The navigation computer 36 and/or the controller 30 may analyze the received RF signals to associate relative states thereto. The RF signals may be of any suitable frequency. The RF transceiver may be positioned at any suitable location to track the objects using RF signals effectively. Furthermore, the RF emitters or transponders may have any suitable structural configuration that may be much different than the trackers as shown in FIG. 1.

In yet another example, the navigation system 32 and/or localizer 44 are electromagnetically based. For example, the navigation system 32 may comprise an EM transceiver coupled to the navigation computer 36. The manipulator 14 and the patient 12 may comprise EM components attached thereto, such as any suitable magnetic tracker, electro-magnetic tracker, inductive tracker, or the like. The trackers may be passive or actively energized. The EM transceiver generates an EM field and generates state signals to the controller 30 based upon EM signals received from the trackers. The navigation computer 36 and/or the controller 30 may analyze the received EM signals to associate relative states thereto. Again, such navigation system 32 examples may have structural configurations that are different than the navigation system 32 configuration as shown throughout the Figures.

In yet another example, the navigation system 32 and/or localizer 44 utilize a machine vision system which includes a video camera coupled to the navigation computer 36. The video camera is configured to locate a physical object in a target space. The physical object has a geometry represented by virtual object data stored by the navigation computer 36. The detected objects may be tools, obstacles, anatomical features, trackers, or the like. The video camera and navigation computer 36 are configured to detect the physical objects using image processing techniques such as pattern, color, or shape recognition, edge detection, pixel analysis, neutral net or deep learning processing, optical character recognition, barcode detection, or the like. The navigation computer 36 can compare the captured images to the virtual object data to identify and track the objects. A tracker may or may not be coupled to the physical object. If trackers are utilized, the machine vision system may also include infrared detectors for tracking the trackers and comparing tracking data to machine vision data. Again, such navigation system 32 examples may have structural configurations that are different than the navigation system 32 configuration as shown throughout the Figures. Examples of machine vision tracking systems can be like that described in U.S. Pat. No. 9,603,665, entitled “Systems and Methods for Establishing Virtual Constraint Boundaries” and/or like that described in U.S. Provisional Patent Application No. 62/698,502, filed Jul. 16, 2018, entitled “Systems and Method for Image Based Registration and Calibration,” the entire contents of which are incorporated by reference herein.

The navigation system 32 and/or localizer 44 may have any other suitable components or structure not specifically recited herein. Furthermore, any of the techniques, methods, and/or components described above with respect to the camera-based navigation system 32 shown throughout the Figures may be implemented or provided for any of the other examples of the navigation system 32 described herein. For example, the navigation system 32 may utilize solely inertial tracking or any combination of tracking techniques.

As shown in FIG. 2, the controller 30 further includes software modules. The software modules may be part of a computer program or programs that operate on the manipulator computer 26, navigation computer 36, or a combination thereof, to process data to assist with control of the system 10. The software modules include instructions stored in one or more non-transitory computer readable medium or memory on the manipulator computer 26, navigation computer 36, or a combination thereof, to be executed by one or more processors of the computers 26, 36. Additionally, software modules for prompting and/or communicating with the operator may form part of the program or programs and may include instructions stored in memory on the manipulator computer 26, navigation computer 36, or a combination thereof. The operator interacts with the first and second input devices 40, 42 and the one or more displays 38 to communicate with the software modules. The user interface software may run on a separate device from the manipulator computer 26 and navigation computer 36.

The controller 30 includes a manipulator controller 60 for processing data to direct motion of the manipulator 14. In one example, as shown in FIG. 1, the manipulator controller 60 is implemented on the manipulator computer 26. The manipulator controller 60 may receive and process data from a single source or multiple sources. The controller 30 further includes a navigation controller 62 for communicating the state data relating to the anatomy to the manipulator 14 to the manipulator controller 60. The manipulator controller 60 receives and processes the state data provided by the navigation controller 62 to direct movement of the manipulator 14. In one example, as shown in FIG. 1, the navigation controller 62 is implemented on the navigation computer 36. The manipulator controller 60 or navigation controller 62 may also communicate states of the patient 12 and manipulator 14 to the operator by displaying an image of the anatomy and the manipulator 14 on the one or more displays 38. The manipulator computer 26 or navigation computer 36 may also command display of instructions or request information using the display 38 to interact with the operator and for directing the manipulator 14.

The one or more controllers 30, including the manipulator controller 60 and navigation controller 62, may be implemented on any suitable device or devices in the system 10, including, but not limited to, the manipulator computer 26, the navigation computer 36, and any combination thereof. As will be described herein, the controller 30 is not limited to one controller, but may include a plurality of controllers for various systems, components, or sub-systems of the surgical system 10. These controllers may be in communication with each other (e.g., directly, or indirectly), and/or with other components of the surgical system 10, such as via physical electrical connections (e.g., a tethered wire harness) and/or via one or more types of wireless communication (e.g., with a WiFi™ network, Bluetooth®, a radio network, and the like). Any of the one or more controllers 30 may be realized as or with various arrangements of computers, processors, control units, and the like, and may comprise discrete components or may be integrated (e.g., sharing hardware, software, inputs, outputs, and the like). Any of the one or more controllers may implement their respective functionality using hardware-only, software-only, or a combination of hardware and software. Examples of hardware include, but is not limited, single or multi-core processors, CPUs, GPUs, integrated circuits, microchips, or ASICs, digital signal processors, microcontrollers, field programmable gate arrays, systems on a chip, discrete circuitry, and/or other suitable hardware, and the like. The one or more controllers may implement software programs, software modules, algorithms, logical rules, look-up tables and other reference data, and various software layers for implementing any of the capabilities described herein. Equivalents of the software and hardware for the one or more controllers 30, and peripheral devices connected thereto, are fully contemplated.

As shown in FIG. 2, the controller 30 includes a boundary generator 66. The boundary generator 66 is a software module that may be implemented on the manipulator controller 60. Alternatively, the boundary generator 66 may be implemented on other components, such as the navigation controller 62. The boundary generator 66 generates virtual boundaries (VB) for constraining the manipulator 14 and/or the surgical tool 20. Such virtual boundaries (VB) may also be referred to as virtual meshes, virtual constraints, line haptics, or the like. The virtual boundaries (VB) may be defined with respect to a 3-D bone model registered to one or more patient trackers such that the virtual boundaries (VB) are fixed relative to the bone model. The state of the manipulator 14 and/or the surgical tool 20 is tracked relative to the virtual boundaries (VB). In one example, the state of a center point of the surgical tool 20 is measured relative to the virtual boundaries (VB) for purposes of determining when and where haptic feedback force is applied to the manipulator 14, or more specifically, the surgical tool 20.

A tool path generator 68 is another software module run by the controller 30, and more specifically, the manipulator controller 60. The tool path generator 68 generates a path for the manipulator 14 and/or the surgical tool 20 to traverse, such as for removing sections of the anatomy to receive an implant. One exemplary system and method for generating the tool path is explained in U.S. Pat. No. 9,119,655, entitled, “Surgical Manipulator Capable of Controlling a Surgical Instrument in Multiple Modes,” the disclosure of which is hereby incorporated by reference. In some examples, the virtual boundaries (VB) and/or tool paths may be generated offline rather than on the manipulator computer 26 or navigation computer 36. Thereafter, the virtual boundaries (VB) and/or tool paths may be utilized at runtime by the manipulator controller 60.

Additionally, it may be desirable to control the manipulator 14 in different modes of operation for the system 10. For example, the system 10 may enable the manipulator 14 to interact with the site using manual and semi-autonomous modes of operation. An example of the semi-autonomous mode is described in U.S. Pat. No. 9,119,655, entitled, “Surgical Manipulator Capable of Controlling a Surgical Instrument in Multiple Modes,” the disclosure of which is hereby incorporated by reference. In the semi-autonomous mode, the manipulator 14 directs movement of the robotic arm 18A and/or the surgical tool 20 at the surgical site. In one instance, the controller 30 models the robotic arm 18A and/or the surgical tool 20 as a virtual rigid body and determines forces and torques to apply to the virtual rigid body to advance and constrain the robotic arm 18A and/or the surgical tool 20 along any trajectory or path in the semi-autonomous mode. Movement of the tool 20 in the semi-autonomous mode is constrained in relation to the virtual constraints generated by the boundary generator 66 and/or path generator 69.

In the semi-autonomous mode, the manipulator 14 is capable of moving the robotic arm 18A and/or surgical tool 20 free of operator assistance. Free of operator assistance may mean that an operator does not physically move the robotic arm 18A and/or surgical tool 20 by applying external force to move the robotic arm 18A and/or surgical tool 20. Instead, the operator may use some form of control to manage starting and stopping of movement. For example, the operator may hold down a button of a control to start movement of the robotic arm 18A and/or surgical tool 20 and release the button to stop movement of the robotic arm 18A and/or surgical tool 20. Alternatively, the operator may press a button to start movement of the robotic arm 18A and/or surgical tool 20 and press a button to stop motorized movement of the robotic arm 18A and/or surgical tool 20 along the trajectory or path. The manipulator 14 uses motorized movement to advance the robotic arm 18A and/or surgical tool 20 in accordance with pre-planned parameters.

Alternatively, the system 10 may be operated in the manual mode, a free mode, or a haptic mode. Here, in one instance, the operator manually directs, and the manipulator 14 controls, movement of the robotic arm 18A and/or surgical tool 20 at the surgical site. The operator physically contacts the robotic arm 18A and/or surgical tool 20 to cause movement of the robotic arm 18A and/or surgical tool 20. In one implementation, the user applies force to cause displacement of the robotic arm 18A and/or surgical tool 20 and the robotic system 10 can reactively provide haptic force feedback when the robotic arm 18A and/or surgical tool 20 reaches certain virtual boundaries. In another implementation, the manipulator 14 monitors the forces and torques placed on the robotic arm 18A and/or surgical tool 20 by the operator in order to position the robotic arm 18A and/or surgical tool 20. A sensor that is part of the manipulator 14, such as a force-torque transducer, measures these external forces and torques applied to the robotic arm 18A and/or surgical tool 20, e.g., in six degrees of freedom. In one example, the sensor is coupled between the distal-most link of the manipulator (J6) and the robotic arm 18A and/or surgical tool 20. In response to the applied forces and torques, the one or more controllers 30, 60, 62 are configured to determine a commanded position of the robotic arm 18A and/or surgical tool 20 by evaluating the forces/torques applied externally to the robotic arm 18A and/or surgical tool 20 with respect to virtual model of the robotic arm 18A and/or surgical tool 20 in a virtual simulation. The manipulator 14 then mechanically moves the robotic arm 18A and/or surgical tool 20 to the commanded position in a manner that emulates the movement that would have occurred based on the forces and torques applied externally by the operator. Movement of the robotic arm 18A and/or surgical tool 20 in the manual mode is also constrained in relation to the virtual constraints generated by the boundary generator 66 and/or path generator 69.

### II. Registration Overview

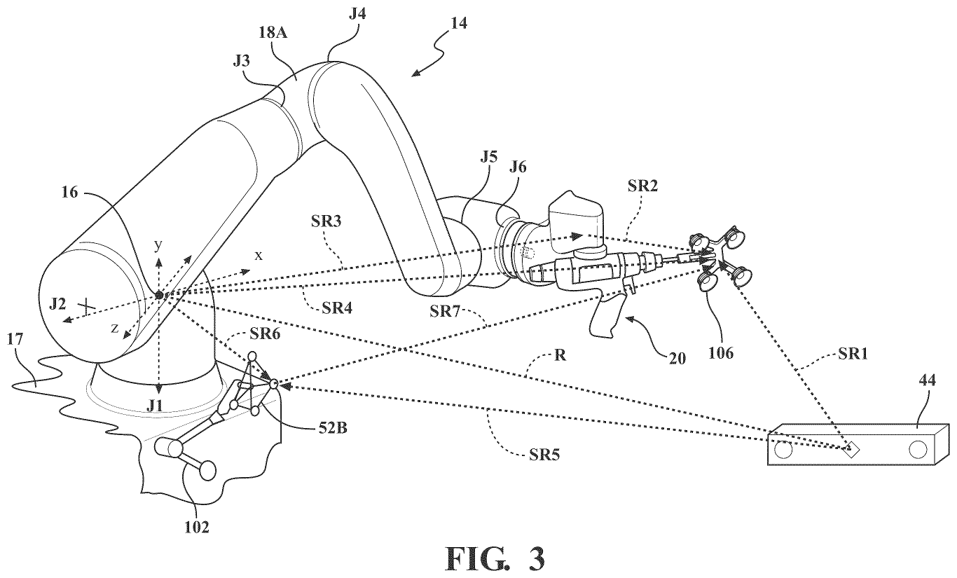

FIG. 3 illustrates transforms that may be utilized in registration of the system 10. During registration of the system 10, the controller 30 establishes a relationship R between the coordinate system of the localizer 44 (i.e., the localizer coordinate system LCLZ) and a coordinate system of the base 16 (i.e., the manipulator coordinate system MNPL).

The controller 30 may obtain a series of sub-relationships between components of the system 10 to establish the relationship R between the localizer coordinate system LCLZ and the manipulator coordinate system MNPL.

The controller 30 may obtain a sub-relationship SR1 between the localizer coordinate system LCLZ and the tool tracker 106 from the localizer 44. For example, the localizer 44 may track the tool tracker 106 and provide a location of the tool tracker 106 to the controller 30 as coordinates in the localizer coordinate system LCLZ.

The controller 30 may obtain a sub-relationship SR2 between the tool tracker 106 and a component of the manipulator 14 from predetermined data. In the instance of FIG. 3, the surgical tool 20 is shown as a distal flange of the robotic arm 18A. The controller 30 may store, in memory, known data regarding a location of the tool tracker 106 relative to the distal flange to obtain the sub-relationship SR2. In some instances, the controller 30 may obtain a sub-relationship SR2 between the tool tracker 106 and the surgical tool 20 from predetermined data. For example, the controller 30 may obtain the sub-relationship SR2 between the tool tracker 106 and the surgical tool 20 based on known data regarding a location of the tool tracker 106 relative to the distal flange and based on known data regarding a location of the distal flange relative to the surgical tool 20. In other instances, the controller 30 may obtain the sub-relationship SR2 between the tool tracker 106 and the surgical tool 20 based on known data regarding a location of the tool tracker 106 relative to the surgical tool 20.

The controller 30 may obtain a sub-relationship SR3 between a component of the manipulator 14 and the manipulator coordinate system MNPL from kinematic data of the manipulator 14. In the instance of FIG. 3, the surgical tool 20 is shown as the distal flange of the robotic arm 18A. The manipulator 14 may sense movement of the distal flange and the controller 30 may receive kinematic data from the manipulator controller 60 characterizing movement of the distal flange. In some instances, the controller 30 may obtain a sub-relationship SR3 between the surgical tool 20 and the manipulator coordinate system MNPL from kinematic data of the manipulator 14. For example, the controller 30 may obtain the sub-relationship SR3 between the surgical tool 20 and the manipulator coordinate system MNPL based on known data regarding a location of the tool tracker 106 relative to the distal flange and based on kinematic data from the manipulator controller 60 characterizing movement of the distal flange. In other instances, the controller 30 may obtain the sub-relationship SR3 between the surgical tool 20 and the manipulator coordinate system MNPL based on kinematic data from the manipulator controller 60 characterizing movement of the surgical tool 20.

For the sub-relationships SR2, SR3, the surgical tool 20 for which the sub-relationships SR2, SR3 are obtained may be any suitable component of the manipulator 14. For example, the surgical tool 20 may be the robotic arm 18A, a link 18 of the robotic arm 18A, a joint (J) of the robotic arm 18A, and/or the surgical tool 20. The controller 30 is able to more accurately establish the relationship R between the localizer coordinate system LCLZ and the manipulator coordinate system MNPL in instances where the surgical tool 20 is the same for both sub-relationships SR2, SR3.

Once the controller 30 obtains a series of sub-relationships between components of the system 10, the controller 30 may establish the relationship R between the localizer coordinate system LCLZ and the manipulator coordinate system MNPL by combining the sub-relationships. For example, the controller 30 may combine sub-relationships SR1, SR2, SR3 to establish the relationship R between the localizer coordinate system LCLZ and the manipulator coordinate system MNPL. Specifically, as shown in FIG. 3, the controller 30 may combine sub-relationships SR2, SR3 to determine the sub-relationship SR4 between the tool tracker 106 and the manipulator coordinate system MNPL. The controller 30 may then combine sub-relationships SR4, SR1 to establish the relationship R between the localizer coordinate system LCLZ and the manipulator coordinate system MNPL.

The controller 30 may additionally establish a sub-relationship SR6 between the base tracker 52B and the manipulator coordinate system MNPL. The controller 30 may determine the sub-relationship SR6 in instances where the tool tracker 106 is a temporary tracker coupled to the surgical tool 20, such as the instance of FIG. 3. In such instances, the tool tracker 106 may be decoupled from the surgical tool 20 during a surgical procedure and the surgical tool 20 may be unable to be tracked by the localizer 44 during the surgical procedure. As such, the relationship R between the localizer coordinate system LCLZ and the manipulator coordinate system MNPL is established in view of the base tracker 52B in order for a location of the surgical tool 20 to be determined during such a surgical procedure. The controller 30 determines the sub-relationship SR6 between the base tracker 52B and the manipulator coordinate system MNPL to establish the relationship R between the localizer coordinate system LCLZ and the manipulator coordinate system MNPL in view of the base tracker 52B. In this way, during a surgical procedure where the tool tracker 106 is removed, the manipulator 14 may determine a location of the surgical tool 20 is determined using kinematic data, where a position of the manipulator 14 is established via the sub-relationship SR6.

In order to establish the sub-relationship SR6 between the base tracker 52B and the manipulator coordinate system MNPL, the controller 30 may first obtain a sub-relationship SR5 between the between the base tracker 52B and the localizer coordinate system LCLZ from the localizer 44. For example, the localizer 44 may track the base tracker 52B and provide a location of the base tracker 52B and the tool tracker 106 to the controller 30 as coordinates in the localizer coordinate system LCLZ.

The controller 30 may then combine the sub-relationships SR1, SR2, SR3, SR5 to establish the sub-relationship SR6 between the base tracker 52B and the manipulator coordinate system MNPL. Specifically, as shown in FIG. 3, the controller 30 may combine sub-relationships SR2, SR3 to determine the sub-relationship SR4 between the tool tracker 106 and the manipulator coordinate system MNPL. The controller 30 may also combine sub-relationships SR1, SR5 to obtain a sub-relationship SR7 between the tool tracker 106 and the base tracker 52B. The controller 30 may then combine sub-relationships SR4, SR7 to establish the sub-relationship SR6 between the base tracker 52B and the manipulator coordinate system MNPL.

The controller 30 may also be configured to verify the accuracy of the established sub-relationship SR6 between the base tracker 52B and the manipulator coordinate system MNPL in instances where the base tracker 52B is coupled to a known location relative to the manipulator 14. For reference, in instances where the base tracker 52B is coupled to the cart 17 by an adjustable support arm 102, a location of the base tracker 52B may be unknown to the controller 30. As such, the controller 30 establishes the sub-relationship SR6 between the base tracker 52B and the manipulator coordinate system MNPL and uses the sub-relationship SR6 when determining a location of the surgical tool 20 during a surgical procedure where the tool tracker 106 is removed. Contrastingly, in instances where the base tracker 52B is coupled to a known location relative to the manipulator 14, the controller 30 may be configured to obtain a predetermined relationship between the base tracker 52B and the manipulator coordinate system MNPL such that, after the controller 30 establishes the sub-relationship SR6, the controller 30 may compare the established sub-relationship SR6 with the predetermined relationship to verify accuracy of the established sub-relationship SR6. The controller 30 may verify the accuracy of the established sub-relationship SR6 as a means of verifying accuracy of the relationship R between the manipulator coordinate system MNPL and the localizer coordinate system LCLZ.

In instances where the base tracker 52B is coupled for effecting continuous movement of the surgical tool 20 and the tool tracker 106 to a known location relative to the manipulator 14, the base tracker 52B may be coupled to any suitable component of the manipulator 14. In one such instance, the base tracker 52B may be coupled to and moveable with the robotic arm 18A of the manipulator 14. In such instances, the controller 30 may be configured to obtain the predetermined relationship between the base tracker 52B and the manipulator coordinate system MNPL from kinematic data related to the manipulator 14. The controller 30 may then compare the established sub-relationship SR1 between the base tracker 52B and the manipulator coordinate system MNPL with the predetermined relationship between the base tracker 52B and the manipulator coordinate system MNPL to verify accuracy of the established sub-relationship SR6. In other instances, the base tracker 52B may be coupled to the cart 17. tracker 52B may be coupled to and moveable with the robotic arm 18A of the manipulator 14. In such instances, the controller 30 may be configured to receive the predetermined relationship between the base tracker 52B and the manipulator coordinate system MNPL. The controller 30 may then compare the established sub-relationship SR1 between the base tracker 52B and the manipulator coordinate system MNPL with the predetermined relationship between the base tracker 52B and the manipulator coordinate system MNPL to verify accuracy of the established sub-relationship SR6.

The controller 30 may be configured to determine any other suitable sub-relationships. For example, in instances where the manipulator 14 includes more than one robotic arm 18A, the controller 30 may determine a sub-relationship between the base tracker 52B and one or more of the robotic arms 18A to determine the relationship R between the localizer coordinate system LCLZ and the manipulator coordinate system MNPL. In such instances, one or more of the robotic arms 18A may be coupled to one or more manipulator trackers 52, which may be tracked by the localizer 44 during registration of patient anatomy. During registration of patient anatomy, the controller 30 may receive a location of the manipulator trackers 52 to determine a location of the one or more robotic arms 18A and, furthermore, to determine the sub-relationship between the manipulator coordinate system MNPL, the localizer coordinate system LCLZ, and/or the base tracker 52B and the one or more robotic arms 18A.

### III. Path Registration

### A. Overview

The controller 30 may be configured to establish the relationship R between the manipulator coordinate system MNPL and the localizer coordinate system LCLZ by performing path registration.

Generally, during path registration, the tool tracker 106 and the surgical tool 20 are moved along a path, with the localizer 44 tracking movement of the tool tracker 106 and the manipulator 14 sensing movement of the surgical tool 20. The controller 30 may then register the manipulator coordinate system MNPL to the localizer coordinate system LCLZ based on localizer data from the localizer 44 and based on the kinematic data from the manipulator 14. Referring to FIG. 3, during path registration, the controller 30 may obtain the sub-relationship SR1 from the localizer 44 as the localizer 44 tracks the tool tracker 106 and provides localizer data related to movement of the tool tracker 106, and the controller 30 may obtain the sub-relationship SR3 from the manipulator 14 as the manipulator 14 senses movement of the surgical tool 20 and provides kinematic data related to movement of the surgical tool 20. As previously stated, the controller 30 may determine the sub-relationship SR2 between the tool tracker 106 and the surgical tool 20 from predetermined data. The controller 30 may then combine the sub-relationships SR1, SR2, SR3 to obtain the relationship R between the manipulator coordinate system MNPL and the localizer coordinate system LCLZ.

### B. Method of Path Registration

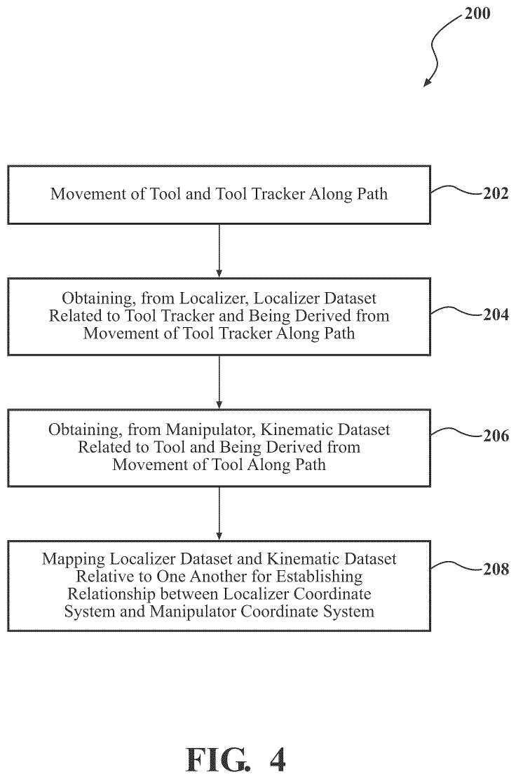

To perform path registration, the controller 30 may execute steps 202-208 of a method 200 of path registration shown in FIG. 4. As shown in FIG. 4, during step 202, the surgical tool 20 and the tool tracker 106 move along a path; during step 204, the controller 30 may obtain, from the localizer 44, a localizer dataset related to the tool tracker 106 and being derived from movement of the tool tracker 106 along the path; during step 206, the controller 30 may obtain, from the manipulator 14, a kinematic dataset related to the surgical tool 20 and being derived from movement of the surgical tool 20 along the path; and during step 208, the controller 30 may map the localizer dataset and the kinematic dataset relative to one another to establish the relationship R between the localizer coordinate system LCLZ and the manipulator coordinate system MNPL.

Steps 202-208 may be ordered in any suitable order. For example, in some instances, steps 202-206 occur concurrently such that the controller 30 may control movement of the surgical tool 20 and the tool tracker 106 while obtaining the localizer dataset and the kinematic dataset. As another example, the controller 30 may obtain the localizer dataset during step 204 after the controller 30 obtains the kinematic dataset during step 206.

### i. MOVEMENT OF SURGICAL TOOL AND TOOL TRACKER

During step 202, the surgical tool 20 and the tool tracker 106 move along a path. The path used during the method 200 of path registration may be a free-form path FFP defined by a user or a predetermined path defined by the controller 30.

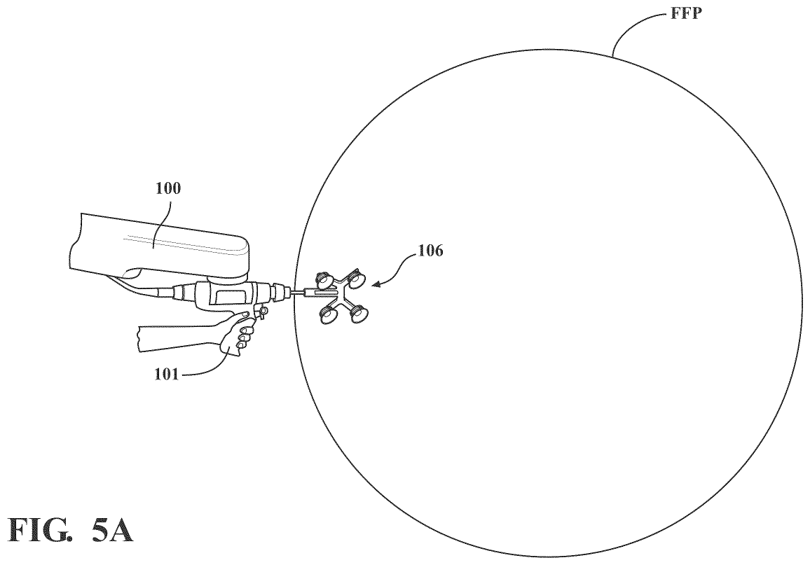

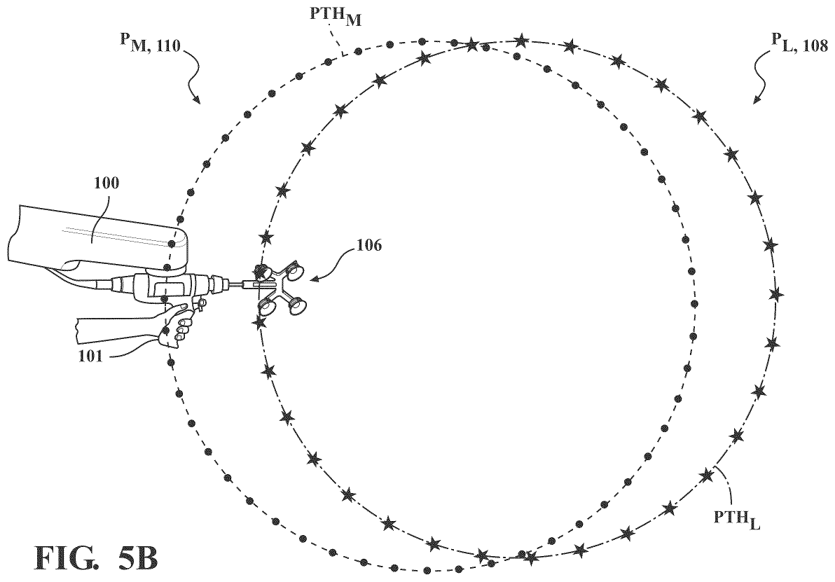

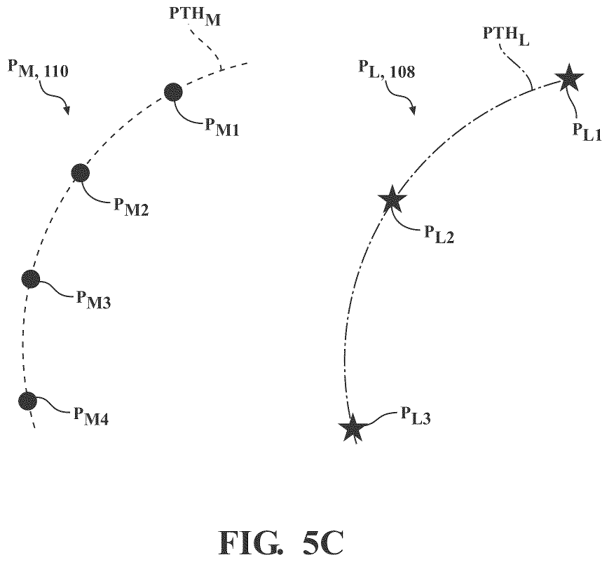

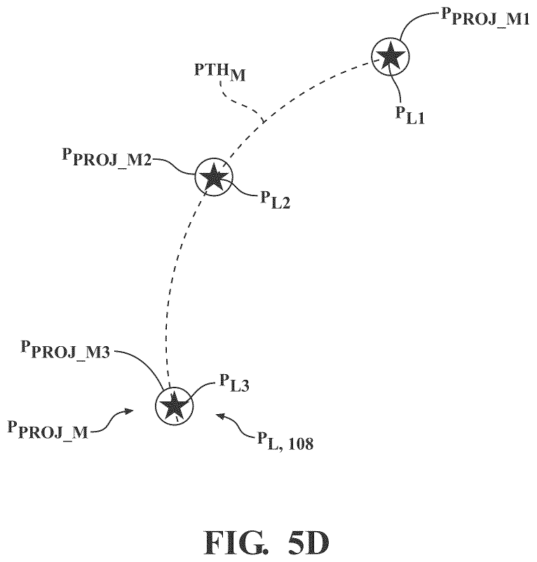

During step 202, the controller 30 may be configured to control, with the robotic arm 18A, movement of the surgical tool 20 and the tool tracker 106 along the path. For example, the controller 30 may be configured to control, with the robotic arm 18A, movement of the surgical tool 20 and the tool tracker 106 in response external force provided by a user. In some instances, the external force may be provided by a user 105 to the robotic arm 18A (as shown in FIG. 1). In some instances, the external force may be provided by a user 105 to the surgical tool 20 (as shown in FIGS. 5A and 5B). The controller 30 receives the external force provided by the user 105 and directs motion of the robotic arm 18A to move the surgical tool 20 and the tool tracker 106 along a path. In other instances, the controller 30 may be configured to control, with the robotic arm 18A, automatic movement of the surgical tool 20 and the tool tracker 106, e.g., along a predetermined path.

In some instances, during step 202, the user 105 may control movement of the surgical tool 20 and the tool tracker 106. For example, in instances where each of the joints (J) are passively driven, the robotic arm 18A may be moved by an external force applied to the manipulator 14. In some instances, the external force may be provided by the user 105 to the robotic arm 18A (as shown in FIG. 1). In some instances, the external force may be provided the user 105 to the surgical tool 20 (as shown in FIGS. 5A and 5B). The user 105 provides external force and the provided external force may move the surgical tool 20 and the tool tracker 106 along the path.

a. Types of Paths

In all instances described herein, the path that the tool tracker 106 and the surgical tool 20 move along may be a free-form path FFP. A free-form path FFP is a path defined by a user in a manner that is unconstrained. For example, the free-form path FFP may be defined by a user without being constrained by virtual constraints generated by the boundary generator 66 and/or path generator 69. An example free-form path FFP is shown in FIG. 5A. In the instance of FIG. 5A, the free-form path FFP is characterized as a circular path. In other instances, the free-form path FFP may be characterized as any continuous two or three-dimensional path for effecting continuous movement of the surgical tool 20 and the tool tracker 106. The free-form path FFP may be characterized as a curved line, a straight line, and/or a combination of curved and straight lines. In some instances, the free-form path FFP may intersect itself.

In some instances, a user may define the free-form path FFP during movement of the tool tracker 106 and the surgical tool 20. In such instances, the free-form path FFP may be unknown prior to movement of the surgical tool 20 and the tool tracker 106. For example, the user 105 may define the free-form path FFP by providing an external force to the manipulator 14 to cause continuous movement of the surgical tool 20 and the tool tracker 106. In such instances, the controller 30 may control, with the robotic arm 18A, movement of the surgical tool 20 of the manipulator 14 and the tool tracker 106 in response to the external force and the resulting path travelled by the surgical tool 20 and the tool tracker 106 defines the free-form path FFP.

In some instances, a user may define the free-form path FFP prior to movement of the tool tracker 106 and the surgical tool 20. For example, the user may define the free-form path FFP using a user interface and/or a computing system to cause continuous movement of the surgical tool 20 and the tool tracker 106. For instance, the user may draw a line on a user interface (e.g. a tablet or a phone), and the line may be used to define the free-form path FFP. In another instance, the user may draw a line on a computing system using a user interface (e.g. a mouse), and the line may be used to define the free-form path FFP.

The user may consider any suitable factor when defining the free-form path FFP. For example, the user may define the free-form path FFP to prevent collisions between the manipulator 14, the surgical tool 20, and/or the tool tracker 106 with objects in the operating room. Additionally, or alternatively, the user may define the free-form path FFP while prioritizing generation of the localizer dataset by the localizer 44, prioritizing generation of the kinematic dataset by the manipulator 14, and/or prioritizing a speed of the path registration method 200. For example, the user may define the free-form path FFP such that the tool tracker 106 remains visible to the localizer 44 during the path registration method 200.

The controller 30 may provide an instruction and/or suggestion to the user regarding the defined free-form path FFP. The instruction and/or suggestion may be provided on a display device of the system 10, such as the display 38 in FIG. 1, and/or a user interface. Additionally, the instruction and/or suggestion may occur prior to, during, or after definition of the free-form path FFP by the user. For example, in instances where the controller 30 provides the instruction and/or suggestion during definition of the free-form path FFP, and the free-form path FFP is defined during movement of the surgical tool 20 and the tool tracker 106, the controller 30 may provide, on the display 38, a directional arrow instructing and/or suggesting the user 105 to move the surgical tool 20 and tool tracker 106 in a particular direction. In instances where the controller 30 provides the instruction and/or suggestion prior to definition of the free-form path FFP, the controller 30 may provide a sample free-form path FFP′ for the user to consider when defining the free-form path FFP. An example of a sample free-form path FFP′ is shown in FIG. 1 on the display 38.

The controller 30 may provide the instruction and/or suggestion based on preventing collisions between the manipulator 14, the surgical tool 20, and/or the tool tracker 106 with objects in the operating room, prioritizing generation of the localizer dataset by the localizer 44, prioritizing generation of the kinematic dataset by the manipulator 14, and/or prioritizing a speed of the path registration method 200. As an example, the controller 30 may provide an instruction and/or suggestion reflecting a completeness of the free-form path FFP based on a threshold level of accuracy. For instance, the threshold level of accuracy may be based on whether the localizer/kinematic dataset generated/to be generated by the localizer 44/manipulator 14 includes a sufficient amount of data points (e.g. localized positions P_{L}/kinematic positions P_{M}, which will be discussed in greater detail below). The threshold level of accuracy may also be based on an amount of time that the surgical tool 20 and tool tracker 106 has been moved/is to be moved along the free-form path FFP. The threshold level of accuracy may also be based on whether the data points (e.g. localized positions P_{L}/kinematic positions P_{M}, which will be discussed in greater detail below) of the localizer/kinematic dataset are sufficiently spaced, or whether the data points are too close to one another. As another example, the controller 30 may provide an instruction and/or suggestion to the user to modify the defined free-form path FFP based on preventing a collision between the manipulator 14, the surgical tool 20, and/or the tool tracker 106 with an object in the operating room.

In all instances described herein, the path that the tool tracker 106 and the surgical tool 20 move along may be a predetermined path. The predetermined path is a path defined by the controller 30. For example, the predetermined path may be generated by the path generator 68 shown in FIG. 2. The predetermined path may be characterized as any continuous two or three-dimensional path for effecting continuous movement of the surgical tool 20 and the tool tracker 106. The predetermined path may be characterized as a curved line, a straight line, and/or a combination of curved and straight lines. In some instances, the predetermined path may intersect itself. In instances where the controller 30 defines the predetermined path, the controller 30 may control, with the robotic arm 18A, automatic movement of the surgical tool 20 and the tool tracker 106 along the predetermined path. Additionally, in instances where the controller 30 defines the predetermined path, the user 105 may apply an external force to the manipulator in accordance with moving the surgical tool 20 and the tool tracker 106 along the predetermined path, and the controller 30 may control, with the robotic arm 18A, movement of the surgical tool 20 and the tool tracker 106 along the predetermined path

In some instances, the predetermined path may be defined prior to or during movement of the surgical tool 20 and the tool tracker 106. For example, the controller 30 may define movement of the surgical tool 20 and the tool tracker 106 during movement of the surgical tool 20 and the tool tracker 106 such that the surgical tool 20 and the tool tracker 106 avoid collisions with objects detected by the localizer 44. As another example, the controller 30 may define movement of the surgical tool 20 and the tool tracker 106 prior to movement of the surgical tool 20 and the tool tracker 106 such that the method of path registration 200 is expedited.

The controller 30 may determine the predetermined path based on any suitable factor. For example, the controller 30 may define the predetermined path to prevent collisions between the manipulator 14, the surgical tool 20, and/or the tool tracker 106 with objects in the operating room. Additionally, or alternatively, the controller 30 may define the predetermined path while prioritizing generation of the localizer dataset by the localizer 44, prioritizing generation of the kinematic dataset by the manipulator 14, and/or prioritizing a speed of the path registration method 200. For example, the controller 30 may define the predetermined path such that the tool tracker 106 remains visible to the localizer 44 during the path registration method 200.

The controller 30 may provide an instruction and/or suggestion to the user regarding the predetermined path. The instruction and/or suggestion may be provided on a display device of the system 10, such as the display 38 in FIG. 1, and/or a user interface. In one example, the controller 30 may provide an instruction and/or suggestion to the user while the user moves the surgical tool 20 and the tool tracker 106 in accordance with the predetermined path. The controller 30 may provide, on the display 38, a directional arrow instructing and/or suggesting the user 105 to move the surgical tool 20 and tool tracker 106 to move the surgical tool 20 and tool tracker 106 in accordance with the predetermined path. The controller 30 may also provide an indication of a deviation of the surgical tool 20 and the tool tracker 106 from the predetermined path.

b. Unconstrained Movement

In some instances, during step 202, movement of the surgical tool 20 and the tool tracker 106 may be unconstrained by virtual boundaries. In such instances, the controller 30 may control, with the robotic arm 18A, movement of the surgical tool 20 and the tool tracker 106 in a free mode. For example, during registration of the system 10, the controller 30 may enter the free mode and disregard virtual boundaries generated by the boundary generator 66. Advantageously, in instances where the user 105 defines the free-form path FFP during movement of the surgical tool 20 and the tool tracker 106, the user 105 may provide external force to the manipulator 14 to define the free-form path FFP in a manner that avoids obstructions in the operating room. Movement of the surgical tool 20 and the tool tracker 106 may also be unconstrained in instances where the controller 30 defines the predetermined path. In such instances, though the predetermined path is defined by the controller 30, movement of the surgical tool 20 and the tool tracker 106 may be unconstrained such that the surgical tool 20 and the tool tracker 106 may deviate from the predetermined path. For example, the user 105 may provide external force to the manipulator 14 in a manner that deviates from the predetermined path to avoid obstructions in the operating room.

In some instances, during step 202, movement of the surgical tool 20 and the tool tracker 106 may be constrained. For example, movement of the surgical tool 20 and the tool tracker 106 may be constrained in instances where the controller 30 defines the predetermined path. In some exemplary instances, the user 105 may provide an external force to the manipulator 14 and movement of the surgical tool 20 and the tool tracker 106 may be constrained to the predetermined path. In some exemplary instances, the controller 30 may control, with the robotic arm 18A, automatic movement of the surgical tool 20 and the tool tracker 106 along the predetermined path such that movement of the surgical tool 20 and the tool tracker 106 are constrained to the predetermined path.

### ii. TRACKING MOVEMENT OF TOOL TRACKER AND SENSING MOVEMENT OF SURGICAL TOOL

The localizer 44 tracks movement of the tool tracker 106 along the path and generates a localizer dataset 108, shown in FIG. 5B. The localizer dataset 108 is related to the tool tracker 106 and is derived from movement of the tool tracker 106 along the path. The localizer dataset 108 may include a plurality of localized positions P_{L} of the tool tracker 106 (illustrated in FIG. 5B as a “★”). The localized positions P_{L} may be derived from movement of the tool tracker 106 along the path. For example, in some instances, each of the localized positions P_{L} may be generated by the localizer 44 as the tool tracker 106 moves along a path. The localizer dataset 108 may additionally, or alternatively, include a localized path PTH_{L} of the tool tracker 106 (illustrated in FIG. 5B as a dot-dash line). The localized path PTH_{L} may be derived from movement of the tool tracker 106 along the path. For example, the localizer 44 may generate localized positions P_{L} as the tool tracker 106 moves along the path and the localizer 44 may then determine the localized path PTH_{L} as a best-fit line based on the localized positions P_{L}. In this way, the localizer 44 may remove noise from the localizer dataset 108 when determining the localized path PTH_{L}, where the noise may be created by random disturbances of the system 10 and/or by outlier localized positions P_{L} (e.g. when the tool tracker 106 is not visible by the localizer 44). Additionally, after the localizer 44 determines the localized path PTH_{L}, the localizer 44 may generate additional localized positions P_{L} based on the determined localized path PTH_{L}. For example, the localizer 44 may sample localized position P_{L} from the determined localized path PTH_{L}.

The manipulator 14 senses movement of the surgical tool 20 along the path and generates a kinematic dataset 110, shown in FIG. 5B. The kinematic dataset 110 is related to the surgical tool 20 and is derived from movement of the surgical tool 20 along the path. The kinematic dataset 110 may include a plurality of kinematic positions P_{M} of the surgical tool 20 (illustrated in FIG. 5B as a “•”). The kinematic positions P_{M} may be derived from movement of the surgical tool 20 along the path. For example, in some instances, each of the kinematic positions P_{M} may be generated by the manipulator 14 as the surgical tool 20 moves along the path. The kinematic dataset 110 may additionally, or alternatively, include a kinematic path PTH_{M} of the surgical tool 20 (illustrated in FIG. 5B as a dot-dash line). The kinematic path PTH_{M} may be derived from movement of the surgical tool 20 along the path. For example, the manipulator 14 may generate kinematic positions P_{M} as the surgical tool 20 moves along the path and the manipulator 14 may then determine the kinematic path PTH_{M} as a best-fit line based on the kinematic positions P_{M}. In this way, the manipulator 14 may remove noise from the kinematic dataset 110 when determining the kinematic path PTH_{M}, where the noise may be created by random disturbances of the system 10 and/or by outlier kinematic positions P_{M}. Additionally, after the manipulator 14 determines the kinematic path PTH_{M}, the manipulator 14 may generate additional kinematic positions P_{M} based on the determined kinematic path PTH_{M}. For example, the manipulator 14 may sample kinematic positions P_{M} from the determined kinematic path PTH_{M}.

The controller 30 obtains the localizer data set 108 and the kinematic dataset 110 during steps 204, 206, respectively. As previously stated, the localizer data set 108 may include the localized positions P_{L} and/or the localized path PTH_{L}. As follows, during steps K04, 206, the controller 30 may obtain the localized positions P_{L} and/or the localized path PTH_{L} from the localizer 44. Similarly, the kinematic data set 110 may include the kinematic positions P_{M} and/or the kinematic path PTH_{M}. As follows, during steps 204, 206, the controller 30 may obtain the kinematic positions P_{M} and/or the kinematic path PTH_{M} from the manipulator 14.

In some instances, the controller 30 may generate the localized positions P_{L}, the localized path PTH_{L}, the kinematic positions P_{M}, and/or the kinematic path PTH_{M} after receiving the localizer dataset 108 during step 204 and after receiving the kinematic dataset 110 during step 206. For example, the controller 30 may receive the localized positions P_{L} included in the localizer dataset 108 and determine the localized path PTH_{L} as a best-fit line based on the received localized positions P_{L}. The controller 30 may also receive the kinematic positions P_{M} included in the kinematic dataset 110 and determine the kinematic path PTH_{M} as a best-fit line based on the received kinematic positions P_{M}. As another example, the controller 30 may generate additional localized positions P_{L} and/or additional kinematic positions P_{M} based on a received or determined localized path PTH_{L} and/or kinematic path PTH_{M}, respectively. For instance, the controller 30 may sample localized positions P_{L} from a received or determined localized path PTH_{L} and the controller 30 may sample kinematic positions P_{M} from a received or determined kinematic path PTH_{M}.

### iii. DETERMINING RELATIONSHIP BETWEEN MANIPULATOR COORDINATE SYSTEM AND LOCALIZER COORDINATE SYSTEM

During step 208, the controller 30 maps the localizer dataset 108 and the kinematic dataset 110 relative to one another to establish the relationship R between the localizer coordinate system LCLZ and the manipulator coordinate system MNPL. The controller 30 may map the localized positions P_{L} and one of or both of the kinematic positions P_{M} and kinematic path PTH_{M} relative to one another. Similarly, the controller 30 may map the localized path P_{L} and one of or both of the kinematic positions P_{M} and kinematic path PTH_{M} relative to one another. For example, in an instance where the controller 30 obtains the localized positions P_{L} and the kinematic positions P_{M} during steps 204, 206, respectively, the controller 30 may map the localized positions P_{L} and kinematic positions P_{M} relative to one another to establish the relationship R between the localizer coordinate system LCLZ and the manipulator coordinate system MNPL. In an instance where the controller 30 obtains the localized path PTH_{L} and the kinematic path PTH_{M} during steps 204, 206, respectively, the controller 30 may map the localized path PTH_{L} and the kinematic path PTH_{M} relative to one another to establish the relationship R between the localizer coordinate system LCLZ and the manipulator coordinate system MNPL.

The controller 30 may map the localizer dataset 108 and the kinematic dataset 110 relative to one another such that the localizer dataset 108 and the kinematic dataset 110 are substantially overlaid. For example, the controller 30 may map the localized positions P_{L} and kinematic positions P_{M} relative to one another by adjusting either the localized positions P_{L} or the kinematic positions P_{M} or both of the localized positions P_{L} and the kinematic positions P_{M} such that the localized positions P_{L} and the kinematic positions P_{M} are substantially overlaid. As another example, the controller 30 may map the localized path PTH_{L} and the kinematic path PTH_{M} relative to one another by adjusting either the localized path PTH_{L} or the kinematic path PTH_{M} or both of the localized path PTH_{L} and the kinematic path PTH_{M} such that the localized path PTH_{L} and the kinematic path PTH_{M} are substantially overlaid. The controller 30 may then establish the relationship R between the localizer coordinate system LCLZ and the manipulator coordinate system MNPL based on the adjustment. In a more specific instance, the controller 30 may establish the relationship R between the localizer coordinate system LCLZ and the manipulator coordinate system MNPL based on the adjustment and based on the sub-relationship SR2 between the tool tracker 106 and the surgical tool 20.

### iv. OTHER CONSIDERATIONS

In some instances, the localizer dataset 108 and the kinematic dataset 110 may note be synchronized in time. For example, the controller 30 may obtain the localizer dataset 108 according to a first sampling rate and the kinematic dataset 110 at a second sampling rate different than the first sampling rate, with the first sampling rate being different than the second sampling rate. As another example, the localized and kinematic datasets 108, 110 may be temporally offset. For instance, the localizer 44 may track movement of the tool tracker 106 after the manipulator 14 begins sensing movement of the surgical tool 20, or the manipulator 14 may begin sensing movement of the surgical tool 20 before the localizer 44 begins tracking movement of the tool tracker 106.

The controller 30 may obtain the localizer dataset 108 according to a first sampling rate and the kinematic dataset 110 at a second sampling rate different than the first sampling rate. In such instances, the localizer 44 may track movement of the tool tracker 106 at the first sampling rate and generate the localized dataset 108 accordingly. For instance, the localizer 44 may generate the localized positions P_{L} during movement of the tool tracker 106 according to the first sampling rate. Similarly, the manipulator 14 may sense movement of the surgical tool 20 at the second sampling rate and generate the kinematic dataset 110 accordingly. For instance, the manipulator 14 may generate the kinematic positions P_{M} at the second sampling rate. In some instances, the second sampling rate may exhibit latency relative to the first sampling rate. For example, the manipulator 14 may sense movement of the surgical tool 20 at a slower rate than the localizer 44 tracks movement of the tool tracker 106. In other instances, the first sampling rate may exhibit latency relative to the second sampling rate. For example, the manipulator 14 may sense movement of the surgical tool 20 at a faster rate than the localizer 44 tracks movement of the tool tracker 106. Such an instance is shown in FIG. 5C. As shown, while the surgical tool 20 and the tool tracker 106 move along the path, the localizer 44 generates three localized positions P_{L1}-P_{L3} and the manipulator 14 generates four kinematic positions P_{M1}-P_{M4}.

The localized and kinematic datasets 108, 110 may be temporally offset. The localizer and kinematic datasets 108, 110 may be generated accordingly such that the localized/kinematic position P_{L}, P_{M} are temporally offset. In such instances, the localized positions PL and the kinematic positions PM are not captured by the localizer 44 and the manipulator 14, respectively, at the same time.

During step 208, the controller 30 may map the localizer dataset 108 and the kinematic dataset 110 relative to one another to offset a latency between the first sampling rate and the second sampling rate and to remove any temporal offset between the localizer dataset 108 and the kinematic dataset 110. In this way, the controller 30 minimizes potential error that may result from the latency and the temporal offset. In instances including a latency between the first sampling rate and the second sampling rate, the localized and kinematic datasets 108, 110 may include a different number of localized and kinematic positions P_{L}, P_{M}. In instances including a temporal offset exists between the localizer dataset 108 and the kinematic dataset 110, the localized and kinematic positions P_{L}, P_{M} may be offset from one another. In both instances, the controller 30 may be configured to offset the latency and remove the temporal offset by mapping projected kinematic/localized positions and the localized/kinematic position P_{L}, P_{M} relative to one another. Instances where a unique localized or kinematic position P_{M}, P_{L} is mapped to a unique projected point may be referred to as “point-to-point” registration.

Referring to FIG. 5D, the controller 30 and/or the manipulator 14 may generate three projected kinematic positions P_{PROJ_M1}-P_{PROJ_M3} corresponding to the three localized positions P_{L1}-P_{L3} of FIG. 5C. The projected kinematic positions P_{PROJ_M1}-P_{PROJ_M3} may be generated as points along the kinematic path PTH_{M}. In some instances, the projected kinematic positions P_{PROJ_M1}-P_{PROJ_M3} may be generated such that, after the localized positions P_{L} and the projected kinematic positions are mapped to one another, a distance between the projected kinematic positions and the corresponding localized positions P_{L} is minimized. FIG. 5D illustrates an instance where a distance between the projected kinematic positions P_{PROJ_M1}-P_{PROJ_M3} and the three localized positions P_{L1}-P_{L3} is minimized after mapping. The controller 30 may then map the projected kinematic positions P_{PROJ_M1}-P_{PROJ_M3} and the three localized positions P_{L1}-P_{L3} to establish the relationship R between the localizer coordinate system LCLZ and the manipulator coordinate system MNPL.

Similarly, the controller 30 may be configured to offset the latency between the first sampling rate and the second sampling rate and to remove any temporal offset between the localizer dataset 108 and the kinematic dataset 110 based on mapping the projected localized positions and the kinematic positions P_{M} relative to one another. The controller 30 and/or the localizer 44 may generate projected localized positions corresponding to the kinematic positions P_{M}.

The projected kinematic/localized positions may be generated as points along the kinematic/localized path PTH_{M}, PTH_{L}, respectively. As previously stated, the kinematic/localized paths PTH_{M}, PTH_{L} may be derived from movement of the tool tracker 106 along the path. Specifically, the kinematic/localized paths PTH_{M}, PTH_{L} may be based on kinematic/localized positions P_{M}, P_{L}. In this way, although projected kinematic/localized positions may be mapped to localized/kinematic positions PTH_{L}, PTH_{M}, respectively, by the controller 30, the kinematic and localized positions P_{M}, P_{L} are still considered by the controller 30 as part of the mapping.

Advantageously, the method 200 of path registration does not require the surgical tool 20 and the tool tracker 106 to be held static by the user at predetermined positions of the surgical tool 20 and the tool tracker 106 defined by the controller 30 for the controller 30 to determine a relationship between the localizer coordinate system LCLZ and the manipulator coordinate system MNPL. In other words, during the method 200, the surgical tool 20 and the tool tracker 106 may move continuously along the previously described free-form path FFP or the previously described predetermined path without being held static at predetermined positions, and the controller 30 is able to determine the relationship between the localizer coordinate system LCLZ and the manipulator coordinate system MNPL. Contrastingly, in instances where the controller 30 defines predetermined positions of the surgical tool 20 and the tool tracker 106, the localizer 44 tracks a position of the tool tracker 106 and the manipulator 14 senses a position of the surgical tool 20 at the predetermined positions. In one such instance, a user of the system causes movement of the surgical tool 20 and the tool tracker 106 along a path where the surgical tool 20 and the tool tracker 106 are statically held at predetermined positions where the localizer 44 is able to track the position of the tool tracker 106 to generate the localizer dataset, and where the manipulator 14 is able to sense the position of the surgical tool 20 to generate the kinematic dataset. However, during the method 200, the controller 30 need not require predetermined positions of the surgical tool 20 and the tool tracker 106 as the localizer 44 is able to track the tool tracker 106 to generate the localizer dataset 108 and the manipulator 14 is able to sense movement of the surgical tool 20 to generate the kinematic dataset 110 during continuous movement of the surgical tool 20 and the tool tracker 106 along a path (e.g. the previously described free-form path FFP or the previously described predetermined path). The controller then maps the localizer dataset 108 and the kinematic dataset 110 relative to one another to establish the relationship R between the localizer coordinate system LCLZ and the base coordinate system MNPL.

The method 200 of path registration may also include any suitable steps described in U.S. patent application Ser. No. 17/513,324, entitled “Robotic Surgical System with Motorized Movement to a Starting Pose for a Registration or Calibration Routine”, which is incorporated herein by reference. For example, the method 200 may additionally include the steps described in the process 500 for providing a motorized movement of the robotic arm to a starting pose for a registration or calibration routine for the robotic arm, as described in U.S. patent application Ser. No. 17/513,324.

Although specific features of various embodiments of the disclosure may be shown in some drawings and not in others, this is for convenience only. In accordance with the principles of the disclosure, any feature of a drawing or other embodiment may be referenced and/or claimed in combination with any feature of any other drawing or embodiment.

This written description uses examples to describe embodiments of the disclosure and also to enable any person skilled in the art to practice the embodiments, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A surgical system comprising:
a manipulator including:
a base establishing a base coordinate system;
a robotic arm coupled to the base and being formed of links and joints; and
a tool coupled to the robotic arm;
a navigation system including:
a base tracker coupled to the manipulator;
a tool tracker configured to be coupled to the tool; and
a localizer configured to track positions of the base tracker and the tool tracker in a localizer coordinate system; and
a control system coupled to the manipulator and the navigation system and being configured to:
control, with the robotic arm, movement of the tool and the tool tracker along a free-form path;
obtain, from the localizer, a localizer dataset related to the tool tracker and being derived from movement of the tool tracker along the free-form path;
obtain, from the manipulator, a kinematic dataset related to the tool and being derived from movement of the tool along the free-form path; and
map the localizer dataset and the kinematic dataset relative to one another to establish a relationship between the localizer coordinate system and the base coordinate system.

2. The surgical system of claim 1, wherein the control system is configured to control, with the robotic arm, movement of the tool and the tool tracker in response external force applied to the tool by a user.

3. The surgical system of claim 2, wherein the free-form path is defined during movement of the tool and the tool tracker by the user, and the free-form path is unknown prior to movement of the tool and the tool tracker.

4. The surgical system of claim 2, wherein the free-form path is defined by continuous movement of the tool and the tool tracker by the user and without requiring the tool and the tool tracker to be held static by the user at predetermined positions defined by the control system.

5. The surgical system of claim 2, further comprising a display device, and wherein the control system is configured to provide, on the display device, an instruction or suggestion for the user for moving the tool.

6. The surgical system of claim 1, wherein the control system is further configured to control, with the robotic arm, movement of the tool and the tool tracker in a free mode whereby movement of the manipulator is unconstrained by a virtual boundary.

7. The surgical system of claim 1, wherein the control system maps the localizer dataset and the kinematic dataset relative to one another by further being configured to:
obtain, from the localizer dataset, a plurality of localized positions of the tool tracker being derived from movement of the tool tracker along the free-form path;
obtain, from the kinematic dataset, a plurality of kinematic positions of the tool being derived from movement of the tool along the free-form path; and
map the localized positions and kinematic positions relative to one another to establish the relationship between the localizer coordinate system and the base coordinate system.

8. The surgical system of claim 1, wherein the control system maps the localizer dataset and the kinematic dataset relative to one another by further being configured to:
obtain, from the localizer dataset, a localized path related to the tool tracker and being derived from movement of the tool tracker along the free-form path;
obtain, from the kinematic dataset, a kinematic path related to the tool and being derived from movement of the tool along the free-form path; and
map the localized path and the kinematic path relative to one another to establish the relationship between the localizer coordinate system and the base coordinate system.

9. The surgical system of claim 1, wherein the control system is further configured to:
obtain the localizer dataset according to a first sampling rate; and
obtain the kinematic dataset according to a second sampling rate being different than the first sampling rate; and
map the localizer dataset and the kinematic dataset relative to one another to offset a latency between the first sampling rate and the second sampling rate.

10. The surgical system of claim 9, wherein the first sampling rate exhibits the latency relative to the second sampling rate.

11. The surgical system of claim 1, wherein the control system establishes the relationship between the localizer coordinate system and the base coordinate system by being configured to:
obtain a first relationship between the localizer coordinate system and the tool tracker from the localizer;
obtain a second relationship between the tool tracker and the tool from predetermined data;
obtain a third relationship between the tool and the base coordinate system from kinematic data of the manipulator; and
combine the first, second, and third relationships.

12. The surgical system of claim 11, wherein the control system is further configured to:
obtain a fourth relationship between the base tracker and the localizer coordinate system from the localizer; and
combine the first, second, third, and fourth relationships to establish a fifth relationship between the base tracker and the base coordinate system.

13. The surgical system of claim 1, wherein:
the manipulator comprises a cart that supports the robotic arm;
the base coordinate system is established relative to the cart; and
the base tracker is coupled to the cart by an adjustable support arm that extends from the cart.

14. The surgical system of claim 12, wherein:
the base coordinate system is determinable relative to the robotic arm; and
the base tracker is coupled to the robotic arm and is moveable with the robotic arm.

15. The surgical system of claim 14, wherein the control system is configured to:
obtain a predetermined relationship between the base tracker and the base coordinate system from kinematic data related to the manipulator; and
compare the established fifth relationship between the base tracker and the base coordinate system with the predetermined relationship between the base tracker and the base coordinate system to verify accuracy of the established fifth relationship.

16. A method of operating a surgical system, the surgical system comprising a manipulator including a base establishing a base coordinate system, a robotic arm coupled to the base and being formed of links and joints, and a tool coupled to the robotic arm, and a navigation system including a base tracker coupled to the manipulator, a tool tracker coupled to the tool, and a localizer configured to track positions of the base tracker and the tool tracker in a localizer coordinate system, and a control system coupled to the manipulator and the navigation system, the method comprising the control system performing the following steps:
controlling, with the robotic arm, movement of the tool and the tool tracker along a free-form path;
obtaining, from the localizer, a localizer dataset related to the tool tracker and being derived from movement of the tool tracker along the free-form path;
obtaining, from the manipulator, a kinematic dataset related to the tool and being derived from movement of the tool along the free-form path; and
mapping the localizer dataset and the kinematic dataset relative to one another for establishing a relationship between the localizer coordinate system and the base coordinate system.

17. The method of claim 16, comprising the control system controlling, with the robotic arm, movement of the tool and the tool tracker in response external force applied to the tool by a user.

18. The method of claim 17, comprising the control system defining the free-form path during movement of the tool and the tool tracker by the user, and wherein the free-form path is unknown prior to movement of the tool and the tool tracker.

19. The method of claim 17, comprising the control system defining the free-form path by continuous movement of the tool and the tool tracker by the user and without requiring the tool and the tool tracker to be held static by the user at predetermined positions defined by the control system.

20. A surgical system comprising:
a manipulator including:
a base establishing a base coordinate system;
an arm coupled to the base and being formed of links and joints; and
a tool coupled to the arm;
a navigation system including:
a base tracker coupled to the manipulator;
a tool tracker configured to be coupled to the tool; and
a localizer configured to track positions of the base tracker and the tool tracker in a localizer coordinate system; and
a control system coupled to the manipulator and the navigation system and being configured to:
obtain, from the localizer, a localizer dataset related to the tool tracker and being derived from movement of the tool tracker along a free-form path;
obtain, from the manipulator, a kinematic dataset related to the tool and being derived from movement of the tool along the free-form path; and
map the localizer dataset and the kinematic dataset relative to one another to establish a relationship between the localizer coordinate system and the base coordinate system.
